(19) United States
(12) Patent Application Publication
(10) Pub. No.: US 2021/0357548 A1
(43) Pub. Date: Nov. 18, 2021

(54) VIRTUAL OSTEOPOROSIS CLINIC

(71) Applicants: Fresenius Medical Care Holdings, Inc., Waltham, MA (US); Fresenius Medical Care Deutschland GmbH, Bad Homburg (DE)

(72) Inventors: David J. JÖRG, Frankfurt Am Main (DE); Doris Helene FÜRTINGER, Frankfurt am Main (DE); Peter KOTANKO, New York, NY (US); Alhaji CHERIF, Kew Gardens, NY (US)

(21) Appl. No.: 15/930,353

(22) Filed: May 12, 2020

(51) Int. Cl. G06F 30/20; A61B 5/00; G16H 50/50; G16H 50/70; G16H 50/20; G16H 10/40; G16H 10/60; G16H 20/10; G16H 70/40; G16H 20/30

(52) U.S. Cl. CPC G06F 30/20; A61B 5/4848; A61B 5/4842; G16H 50/50; G16H 50/70; G06F 2111/10; G16H 10/40; G16H 10/60; G16H 20/10; G16H 70/40; G16H 20/30; G16H 50/20

(57) ABSTRACT

In one aspect, a method for optimizing a therapy for osteoporosis is disclosed, which comprises (a) generating in silico a plurality of virtual patients, wherein each of the virtual patients comprises a mathematical construct for modeling progression of osteoporosis via simulation of bone remodeling, said mathematical construct comprising a plurality of dynamic mathematical relations defining time-dependent evolution of at least one of a cell density variable or concentration variable associated with any of pre-osteoblasts, osteoblasts, preosteoclasts, osteoclasts, osteocytes, a bone resorption signal, sclerostin, estrogen, bone density and bone mineral content and employs a processor to determine time-variation of at least one of bone mineral density and bone mineral fraction based on at least a portion of said time-dependent variables, (b) applying a simulated therapy to said plurality of virtual patients, and (c) using said virtual patients to determine an effect of said simulated therapy on progression of osteoporosis.

### BACKGROUND

The present teachings are generally related to methods and systems for performing in-silico modeling of bone remodeling, and in particular, methods and systems for in-silico modeling of treatment and progression of osteoporosis.

Bone remodeling is a process by which bone strength and homeostatic function of inorganic components are maintained. The bone remodeling involves replacing portions of the old bone with newly formed bone matrix. Osteoclasts are responsible for bone resorption and osteoblasts are responsible for secreting new bone matrix in a process called ossification. Osteocytes are essentially osteoblasts surrounded by the bone matrix.

Osteoporosis is a disease in which bone density (quantitated as bone mineral density) deceases and results in a porous bone matrix that is more susceptible to fracture. Osteoporosis can result from an increase in bone matrix resorption and/or a decrease in the formation of new bone matrix. A variety of factors can affect the progression of osteoporosis. Some examples of such factors include the age of onset of menopause, age, medication, physical activity, among others. For example, bone resorption rates are much higher in post-menopausal women due to estrogen deficiency caused by menopause. Common treatments of osteoporosis include the administration of drugs that increase bone mineral density. Some examples of such drugs include bisphosphonates, RANKL inhibitors, and selective estrogen receptor modulators (SERMs). Osteopenia is characterized by reduced bone mass of lesser severity than osteoporosis, and can be precursor of osteoporosis.

It is important to know the effect of certain physiological conditions as well as certain therapeutic protocols on the treatment and progression of osteoporosis. Clinical trials can be performed to obtain such information. But conducting a clinical trial is usually expensive and time-consuming.

Accordingly, there is a need for cost-effective, predictive models that can be used to obtain information regarding bone remodeling, and in particular, the effects of various physiological conditions and therapeutic protocols on the treatment and progression of osteoporosis.

### SUMMARY

In one aspect, a method for optimizing a therapy for osteoporosis is disclosed, which comprises (a) generating in silico a plurality of virtual patients, wherein each of the virtual patients comprises a mathematical construct for modeling progression of osteoporosis via simulation of bone remodeling, where the mathematical construct comprises a plurality of dynamic mathematical relations defining time-dependent evolution of at least one of a cell density variable or concentration variable (e.g., cell population) associated with any of pre-osteoblasts, osteoblasts, preosteoclasts, osteoclasts, osteocytes, and a bone resorption signal, sclerostin, estrogen, bone density and bone mineral content and employs a processor to determine time-variation of at least one of bone mineral density and bone mineral fraction based on at least a portion of said time-dependent variables, (b) applying a simulated therapy to said plurality of virtual patients, and (c) using said virtual patients to determine an effect of said simulated therapy on progression of osteoporosis.

In some embodiments, the method can further include: (d) adjusting the simulated therapy by adjusting at least one of the parameters of at least one of the dynamic mathematical relations, (e) applying said adjusted simulated therapy to said virtual patients, and (f) determining an effect of said adjusted simulated therapy on the progression of osteoporosis.

In some embodiments, the above steps d)-f) can be repeated so as to optimize the simulated therapy.

In some embodiments, at least one parameter of at least one of the dynamic mathematical relations is determined based on physiological data collected from a population of actual patients. By way of example, the physiological data can include any of measured bone mineral density, and measured serum concentration of one or more bone turnover markers. In some embodiments, one or more bone turnover markers comprise any of N-terminal propeptide of type I procollagen (PINP), bone-specific alkaline phosphatase (BSAP), and C-terminal telopeptide (CTX).

In some embodiments, the simulated therapy can include the virtual administration of at least one medication to the virtual patients. In some such embodiments, the simulated therapy includes the virtual administration of a plurality of medications (e.g., two or more medications) to the virtual patients. In some such embodiments, the pharmacokinetic data associated with the one or more medications is employed to simulate the effect of those medications (e.g., individually or in combination) on the progression of osteoporosis. Further, in some such embodiments, the effect of the simulated therapy on the progression of osteoporosis can be determined based on a particular dosage and/or regimen employed for the administration of one or more medications to the virtual patients.

In some embodiments, the simulated therapy includes adjusting a dosage of at least one medication (or multiple medications) that is virtually administered to the virtual patients. For example, such adjustment of the medication can include adjusting any of the dose and/or the daily schedule for the virtual administration of the medication(s) to the virtual patients.

Further, in some embodiments, at least one of the virtual patients is configured to simulate an effect of a disease on the progression of the osteoporosis. By way of example, the disease can be multiple myeloma. In some such embodiments, at least one of the virtual patients simulates the effect of multiple myeloma on the progression of osteoporosis by adjusting at least one of the following rates: a birth rate, a differentiation rate or cell death rate of at least one of pre-osteoblasts, pre-osteoclasts, osteoblasts, osteoclasts, and osteocytes. In some such embodiments, the step of adjusting at least one of the rates includes defining a mathematical relation indicative of time variation of the rate after onset of the disease.

In another embodiment, the effect of glucocorticoid-induced osteoporosis on the progression of osteoporosis can be simulated by adjusting at least one of the following rates: a birth rate, a differentiation rate or cell death rate of at least one of pre-osteoblasts, pre-osteoclasts, osteoblasts, osteoclasts, and osteocytes.

In some embodiments, at least one of the variables can be adjusted to simulate an effect of a change in a physiological condition on the progression of osteoporosis. The physiological condition can be, for example, the onset of menopause. In some embodiments, the estrogen concentration can be used as the interface variable for simulating the effect of menopause on the progression of osteoporosis. In some such embodiments, the simulation of the effect of menopause on the progression of osteoporosis can be performed by using a time-dependent relation defining post-menopausal decline of estrogen as a function of time.

In some embodiments, the physiological condition is caused by aging. In some embodiments, the variable associated with the age-dependent physiological condition comprises the serum concentration of sclerostin. By way of example, the simulation of the effect of aging can be achieved by providing a relation that defines a time-dependent increase in the serum concentration of the sclerostin after a selected age threshold.

In some embodiments, the method can further include adjusting at least one of the dynamic variables to simulate the effect of application of a therapy on the progression of osteoporosis. In one embodiment, the therapy includes the virtual administration of a RANKL inhibitor to an individual and the step of adjusting at least one variable includes defining a mathematical relation indicative of time variation of any of the pre-osteoclast cell density, the osteoclast cell density and the bone mineral content as a function of the virtual administration of the RANKL inhibitor.

Further, in some embodiments, the therapy includes the virtual administration of a sclerostin inhibitor and the step of adjusting at least one of the dynamic variables includes defining a mathematical relation that is indicative of time variation of the sclerostin concentration as a function of the virtual administration of the Sclerostin inhibitor.

In some embodiments, the therapy includes the virtual administration of bisphosphonates and the step of adjusting at least one variable comprises defining a mathematical relation indicative of time variation of the osteoclasts cell density as a function of enhanced apoptosis rate due to the virtual administration of bisphosphonates. By way of example, the bisphosphonates can be, without limitation, any of alendronate, risedronate, ibandronate, and zoledronate.

In some embodiments, the method includes adjusting at least one of the dynamic variables to simulate the effect of a physical activity and/or microgravity on any of treatment and progression of osteoporosis. In some such embodiments, the at least one variable associated with the physical activity includes a concentration of the sclerostin and an in-silico model according to the present teachings provides a mathematical relation indicating a temporal change in the concentration of sclerostin as a function of time due to physical activity, as discussed in more detail below. In a related aspect, a method for optimizing a therapy for osteoporosis is disclosed, which includes: (a) generating in silico at least one virtual patient as a mathematical construct for modeling age-related progression of osteoporosis via simulation of bone remodeling, said mathematical construct comprising a plurality of dynamic mathematical relations defining time-dependent evolution of at least one of a cell density variable or concentration variable associated with any of pre-osteoblasts, osteoblasts, preosteoclasts, osteoclasts, osteocytes, a bone resorption signal, sclerostin, estrogen, bone density and bone mineral content and employs a processor to determine time-variation of bone mineral density based on at least a portion of said time-dependent variables, (b) applying a simulated therapy to said at least one virtual patient, and (c) using said at least one virtual patient to determine an effect of said simulated therapy on progression of osteoporosis. In some embodiments, the at least one virtual patient includes a plurality of virtual patients.

In some embodiments, the mathematical construct employs a relation indicative of variation of any of estrogen and sclerostin as a function of age to determine the effect of aging on at least one of said variables. By way of example, such a relation can be as follows:

$$e ( a ) = \{ \begin{matrix}1 , & a < {a}_{e} \\ \frac{1}{1 + ( a - {a}_{e} ) / {τ}_{e}} , & a \geq {a}_{e}\end{matrix}$$

where aₑ denotes the age at the onset of estrogen decline and τₑ is a characteristic time scale.

In some embodiments, the above method can further include adjusting the simulated therapy by adjusting at least one of said parameters of at least one of the dynamic mathematical relations, applying the adjusted simulated therapy to the virtual patients, and determining an effect of the adjusted simulated therapy on the progression of osteoporosis. This process can be iterated so as to obtain an optimal therapy for administration to the patient.

In some embodiments, at least one parameter of at least one of the dynamic mathematical relations is determined based on physiological data collected from a population of actual patients.

In some embodiments, the physiological data include any of measured bone mineral density and measured serum concentration of one or more bone turnover markers. Some examples of such markers include, without limitation, any of N-terminal propeptide of type I procollagen (PINP), bone-specific alkaline phosphatase (B SAP), and C-terminal telopeptide (CTX).

In some embodiments, the simulated therapy includes the virtual administration of at least one medication to the virtual patients. In some such embodiments, the simulated therapy includes the virtual administration of a combination of two or more medications to the virtual patients. In some embodiments, pharmacokinetic data associated with at least one medication is employed for determining the effect of a simulated therapy on the progression of osteoporosis.

In some embodiments, the adjustment of the simulated therapy includes adjusting a dose of at least one medication administered to the virtual patients. For example, the dose and/or the regimen of the administration of the medication to the virtual patients can be modified and the effect of the modification on the progression of osteoporosis can be simulated.

In a related aspect, a computer system for modeling osteoporosis is disclosed, which comprises a simulation module using at least one computer processor for generating in silico a mathematical construct comprising a plurality of dynamic mathematical relations for simulating bone remodeling, where the dynamic mathematical relations define time-dependent evolution of at least one of a density variable or a concentration variable associated with any of pre-osteoblasts, osteoblasts, pre-osteoclasts, osteoclasts, osteocytes, a bone resorption signal, sclerostin, estrogen, bone density and bone mineral content, said simulation module further being configured to determine time-variation of bone mineral density based on at least a portion of the time-dependent variables. The system can further include an avatar module for storing patient-specific parameters and being in communication with the simulation module to provide the patient-specific parameters thereto so as to enable the simulation module to simulate the effect of any of aging, physical activity, a physiological condition, a therapeutic treatment, a disease, a dietary or substance intake on progression of osteoporosis, and a fitting module configured to derive said patient-specific parameters by fitting one or more observables to the clinical data. By way of example, the clinical data can be based on data collected from a population of patients.

In some embodiments, the observables can include any of bone mineral density and bone turnover markers.

In some embodiments, the simulation module is configured to simulate the effect of multiple myeloma on the progression of osteoporosis. For example, the simulation module can simulate the effect of multiple myeloma on the progression of osteoporosis by adjusting a reference differentiation rate of the pre-osteoclasts. The adjustment of the reference differentiation rate includes defining a mathematical relation indicative of time variation of the reference differentiation rate after the onset of the disease.

In some embodiments, the simulation module is configured to simulate the effect of glucocorticoid-induced osteoporosis on the progression of osteoporosis by adjusting a reference differentiation rate of the pre-osteoclasts. In some embodiments, the physiological condition is onset of menopause, and the associated dynamic variable for simulating the effect of the onset of menopause on the progression of osteoporosis is the estrogen concentration. In some such embodiments, the simulation module employs a time-dependent relation defining post-menopausal decline of estrogen as a function of time to simulate the effect of menopause on the progression of osteoporosis.

In some embodiments, the simulation module is configured to adjust a concentration of sclerostin to simulate the effect of aging on the progression of osteoporosis. In some such embodiments, the simulation module employs a relation defining time-dependent increase in the serum concentration of the sclerostin after a selected age threshold to simulate the effect of aging on the progression of osteoporosis.

In some embodiments, the simulation module is configured to simulate the effect of the administration of a RANKL inhibitor on the progression of osteoporosis. In some such embodiments, the simulation module performs the simulation by defining a mathematical relation that is indicative of time variation of any of the pre-osteoclast cell density, the osteoclast cell density and the bone mineral content as a function of the administration of the RANKL inhibitor.

In some embodiments, the therapeutic treatment comprises the administration of a sclerostin inhibitor and the step of adjusting at least one variable comprises defining a mathematical relation indicative of time variation of the sclerostin concentration as a function of the virtual administration of the sclerostin inhibitor.

In some embodiments, the therapeutic treatment includes the administration of bisphosphonates and the step of adjusting at least one variable includes defining a mathematical relation indicative of time variation of the osteoclasts cell density as a function of enhanced apoptosis rate due to the virtual administration of bisphosphonates.

In a related aspect, a computer system for modeling osteoporosis is disclosed, which comprises a fitting module, an avatar module, and a simulation module. The fitting module is configured to receive any of patient-level and population-level clinical data. The fitting module also includes at least one cost function for determining whether a set of calibration parameters associated with the avatar module are acceptable, as discussed in more detail below. The fitting module is further configured to define fit parameters and parameter bounds and communicate this information to the avatar module. The avatar module is in turn configured to pass this information to the simulation module, which is configured to employ the dynamic mathematical relations of an in-silico model according to the present teachings to compute time evolution of one or more variables associated with bone remodeling and pass this information back to the avatar module.

The avatar module employs this information to compute one or more avatar-specific quantities, such as time-dependence of the bone turnover markers. The avatar module compiles the information received from the simulation module and the information it itself computes and transmits the compiled information to the fitting module.

The fitting module employs the compiled information to compute a value of the cost function and compares a reduction in the value of the cost function in each iteration with a predefined threshold. If the value is less than the threshold, the avatar calibration is complete and the fitting module can invoke the calibrated avatar module for obtaining time evolution of parameters of interest. If the value is greater than the threshold, the fitting modules adjusts the avatar parameters and passes the new parameters to the avatar module. This process can be reiterated until the value of the cost function is within an acceptable range.

### BRIEF DESCRIPTION OF THE DRAWINGS

FIG. 1 schematically depicts an in-silico model according to an embodiment of the present teachings for simulating bone mineralization,

FIG. 2 schematically depicts a system according to an embodiment of the present teachings for implementing an in-silico model according to the present teachings,

FIG. 3A is a flow chart depicting various steps in a method according to an embodiment in which one or more avatars created based on clinical data are employed to obtain an optimal osteoporosis treatment,

FIG. 3B is a flow chart depicting the exchange of information among various modules of a system according to an embodiment of the present teachings depicted in FIG. 2,

FIG. 3C schematically depicts an example of an implementation of a hardware system, which can be used to execute the modules depicted in FIG. 2, and

FIGS. 4-48 schematically depict the results of examples of in-silico simulations in accordance with embodiments of the present teachings.

### DETAILED DESCRIPTION

The present teachings are generally directed to methods and systems for in-silico modeling of bone remodeling, and in particular the progression and/or treatment of osteoporosis, which is a medical condition characterized by a decrease in bone mass with decreased density and enlargement of bone spaces producing porosity and fragility.

Various terms are used herein in accordance with their ordinary meanings in the art. The term “about” as used herein denotes a variation of at most 10% around a numerical value and the term “substantially” as used herein denotes a deviation of at most 10% with respect to a complete state and/or condition. The term “osteoporosis” as used herein is intended to encompass any bone loss disease, and illness condition including, without limitation, osteopenia and more advanced stages of bone loss that are generally referred to in medicine as osteoporosis.

As discussed in more detail below, in some embodiments, the modeling of osteoporosis employs a mathematical construct implemented on a computing device, where in some embodiments, the mathematical construct includes a plurality of dynamic mathematical relations that define time-dependent evolution of at least one of a cell density variable or a cell population variable associated with any of pre-osteoblasts, osteoblasts, osteocytes, or a variable indicative of a bone resorption signal, sclerostin and/or estrogen concentration, bone density and bone mineral content.

In some embodiments, the mathematical construct includes only those components of the bone mineral metabolism that is relevant to disease progression and intervention. In particular, in many embodiments, the present teachings employ key modulators that are especially important in the context of osteoporosis (e.g., sex hormones and medication).

In many embodiments, concentrations and densities of various components of the model (e.g., osteoblasts, osteoclasts, etc.) are normalized to steady state healthy adult values in order to provide a non-dimensional model. In some embodiments, a number of time constants (e.g., equilibrium rates encoded in the degradation/apoptosis rates) can be inferred from known values in the literature. Subsequently, the gain rates can be determined self-consistently by imposing a unit steady state that represents the average healthy adult condition.

For self-consistency, in some embodiments, all components that do have a regulatory effect working in both directions are assigned threshold concentrations that are in the range of the steady-state value within an order of magnitude.

Further, in many embodiments, the effects of external factors on the RANK-RANKL-OPG pathway are taken into account by their effective action on the respective cell populations.

As discussed in more detail below, the following features, among others, are incorporated in an in-silica model according to the present teachings for bone mineralization/osteoporosis.

### RANK-RANKL-OPG Pathway

RANK-RANKL-OPG pathway is central to balancing bone formation and bone resorption. RANKL, is a ligand that can bind to RANK receptor of osteoclasts to stimulate them, thereby causing bone resorption. OPG (osteoprotegerin) is secreted by osteoblasts and binds to RANKL to prevent it from binding to RANK receptor, thereby inhibiting excessive bone resorption.

### Bone Morphogenic Unit

In bone matrix, several growth factors are present in a latent form. Examples of such growth factors include TGFβ, BMF2, PDGF and IGFs. These growth factors are deposited by the osteoblasts upon bone formation and are released by osteoclasts upon bone resorption. It is likely that they act on osteoblast progenitor populations. Estrogen inhibits bone resorption by directly inducing apoptosis of the bone-resorbing osteoclasts, among other processes. For example, estrogen is also known to regulate sclerostin, pre-osteoclast differentiation, and osteoblast apoptosis.

### Sclerostin

Sclerostin is a small protein expressed by the SOST gene in osteocytes. When sclerostin binds to its receptors on the cell surface of osteoblasts, a downstream cascade of intracellular signaling is initiated to inhibit bone formation. Circulating levels of sclerostin are regulated by estrogen. From the age 20 to 80, sclerostin serum levels can increase up to about fourfold. However, sclerostin mRNA levels in bone show no significant age dependence. In postmenopausal women, the serum level of sclerostin increases with a concomitant decrease in bone density. In postmenopausal women, the order of magnitude of serum sclerostin levels is about 0.3 ng/ml.

### Overview

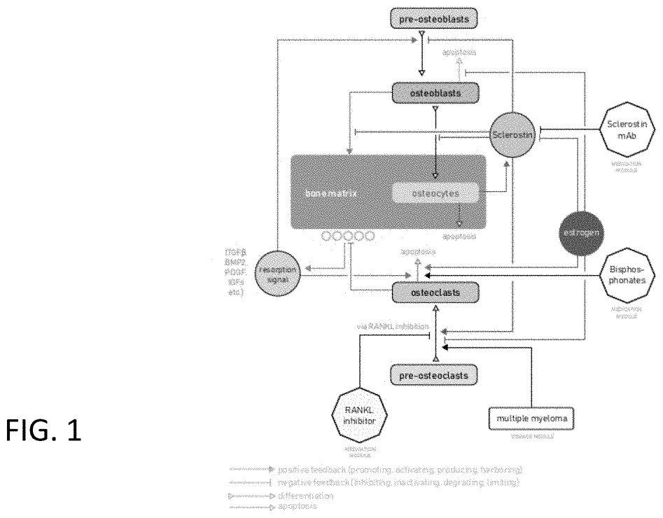

FIG. 1 schematically depicts an overview of an in-silico model according to an embodiment of the present teachings for modeling bone mineralization, and particularly, the progression of osteoporosis, that can be implemented on a digital signal processing system, and which will be discussed in more detail below. In this embodiment, the components of the model include, without limitation, pre-osteoblasts, osteoblasts, pre-osteoclasts, osteoclasts, osteocytes, sclerostin, resorption signal, estrogen, and physical activity.

As discussed in more detail below, the model (and associated in silico system) can provide modules for simulating the effect of a disease and/or a therapeutic treatment on the bone mineralization. By way of example, FIG. 1 schematically shows that the mode can include a module for determining the effect of multiple myeloma on bone mineralization, as discussed in more detail below. Further, modules for determining the effect of virtual administration of one or more therapeutic agents can be provided. In particular, such modules can be added to the core model by providing an interface to one or more dynamic mathematical relations of the core model, which describe the time evolution of one or more model components. By way of example, and without limitation, FIG. 1 schematically shows that a model according to the present teachings can include modules for determining the effect of virtual administration of RANKL inhibitors, bisphosphonates, sclerostin mAbs (monoclonal antibodies) on bone mineralization.

In the embodiments discussed below, the following conventions and abbreviations are employed.

### Symbols

ρ—dynamic cell density variables
θ—external concentrations
μ—reference gain rates
κ—rates of first-order degradation
ω—differentiation rate
η—apoptosis rate
ν—scaling factors weighing different contributions to the same effect
Γ⁺, Γ⁻—sigmoidal positive/negative feedback functions

$${Γ}^{+} ( x ) = \frac{{x}^{n}}{1 + {x}^{n}} , \\ {Γ}^{-} ( x ) = \frac{1}{1 + {x}^{n}}$$

### Component Labels

B*—Pre-osteoblasts
B—Osteoblasts
Y—Osteocytes
C*—Pre-osteoclasts
C—Osteoclasts

Superscripts indicate components; e.g., ρ^{B} denotes osteoblast density. Regulatory threshold concentrations are denoted by the same symbol as the dynamic variable with a subscript indicating on which component the regulation acts; e.g., s_{B} is the threshold sclerostin concentration for the regulation of osteoblasts (B).

### Other Densities

s—sclerostin
e—estrogen
ϕ—hone mineral content
Ω—bone density
Ωₘ—bone mineral density

### Units

In the discussion below, the following units are employed:
The unit of time is days.
The steady state of each concentration and density variable (φ, ρ, θ, etc.) represents the healthy adult value (e.g., at 20-30 years of age). Hence, all parameters either have the dimension of arbitrary concentration/density units per time, inverse time or are dimensionless.

### Osteoblast Lineage

In some embodiments, systems and methods according to the present teachings for modeling bone mineralization employ a plurality of dynamic mathematical relations for determining the time evolution of any of concentration and density of various components of the model.

In some embodiments, bone remodeling can be modeled in silica by defining a plurality of dynamic mathematical relations that incorporate the following information regarding the transitions of pre-osteoblasts to osteoblasts and osteoblasts to osteocytes: (a) pre-osteoblast to osteoblast differentiation is upregulated by the resorption signal and downregulated by sclerostin; (b) osteoblast apoptosis is downregulated by estrogen; (c) osteoblast to osteocyte transition is downregulated by sclerostin; (d) osteocytes are exclusively generated via transition from osteoblasts and undergo apoptosis at a constant rate, and (e) osteoblast to osteocyte transition is downregulated by sclerostin. More specifically, in an embodiment of an in-silico model according to the present teachings, the following relation defines the pre-osteoblast to osteoblast flux:

$${f}_{B * \to B} = {Γ}^{-} ( \frac{s}{{s}_{B *}} ) s [ 1 + {v}^{B *} {Γ}^{+} ( \frac{r}{{r}_{B}} ) ] {ω}^{B *} {ρ}^{B *} \qquad Eq . ( 1 ) ( pre \text{-} osteoblast to osteoblast flux )$$

The following relation defines the osteoblast to osteocyte flux:

$${f}_{B \to Y} = {Γ}^{-} ( \frac{s}{{s}_{Y}} ) {ω}^{B} {ρ}^{B} \qquad Eq . ( 2 ) ( osteoblast to osteocyte flux )$$

The following relation defines the pre-osteoblast dynamics:

\dot{ρ}^{B*}=μ^{B*}−f_{B*→B} Eq. (3) (pre-osteoblast dynamics)

The following relation defines the osteoblast dynamics:

$${\dot{ρ}}^{B} = {α}^{B *} {f}_{B * \to B} - {Γ}^{-} ( \frac{e}{{e}_{B}} ) {η}^{B} {ρ}^{B} - {f}_{B \to Y} \qquad Eq . ( 4 ) ( osteoblast dynamics )$$

The following relation defines the osteocyte dynamics:

\dot{ρ}^{Y}=f_{B→Y}−η^{Y}ρ^{Y} Eq. (5) (osteocyte dynamics)

### Osteoclast Lineage

Osteoclasts are multi-nucleated cells that contain many mitochondria and lysosomes. Osteoclasts cause bone resorption by secreting enzymes, such as collagenase, to release minerals, such as calcium, magnesium, and phosphate into the extracellular fluid.

In some embodiments, the following dynamic mathematical relations simulate the dynamics of the osteoclast lineage by taking into account that (a) the pre-osteoclast to osteoclast differentiation is upregulated by RANKL bound to (osteoclastic) RANK and (b) osteoclast apoptosis is upregulated by estrogen and the resorption signal.

In an embodiment of an in-silico model according to the present teachings, the following relation defines the pre-osteoclast to osteoclast flux:

$${f}_{C * \to C} = {Γ}^{-} ( \frac{e}{{e}_{C *}} ) {Γ}^{+} ( \frac{s}{{s}_{C *}} ) {ω}^{C *} {ρ}^{C *} \qquad Eq . ( 6 ) ( pre \text{-} osteoclast to osteoclast flux )$$

The following relation defines the pre-osteoclast dynamics:

\dot{ρ}^{C*}=μ^{C*}−f_{C*→C} Eq. (7) (pre-osteoclast dynamics)

The following relation defines the osteoclast dynamics:

$${\dot{ρ}}^{C} = {α}^{C *} {f}_{C * \to C} - [ 1 + {v}^{C} {Γ}^{+} ( \frac{e}{{e}_{C}} ) {Γ}^{+} ( \frac{r}{{r}_{C}} ) ] {η}^{C} {ρ}^{C} \qquad Eq . ( 8 ) ( osteoclast dynamics )$$

### Sclerostin

The systems and methods according to the present teachings also incorporate the role of sclerostin in an in-silico model of hone mineralization. Sclerostin is a protein that is encoded by the SOST gene. More specifically, sclerostin is a secreted glycoprotein with a C-terminal cysteine knot-like (CTCK) domain and sequence similarity to the DAN (differential screening-selected gene aberrative in neuroblastoma) family of bone morphogenetic protein (BMP) antagonists.

Sclerostin is produced by osteocytes and undergoes independent degradation. Sclerostin production intensity by osteocytes depends on time. Further, sclerostin production is downregulated by estrogen. The following dynamic mathematical relation simulates the estrogen dynamics by incorporating the above information.

In some embodiments, the present teachings take into account the concentration of sclerostin in the simulation of bone mineralization. Sclerostin is produced by osteocytes and its main function is to inhibit bone formation. In particular, sclerostin exerts its effects mainly by interfering with a process called Writ signaling, which plays an important role in the regulation of bone formation.

Sclerostin is produced by osteocytes and undergoes independent degradation. The intensity of sclerostin production by osteocytes depends on time. Further, sclerostin production is downregulated by estrogen. In some embodiments, the following relation is employed to define the rate of change of the sclerostin density as a function of time:

$$\dot{s} = {μ}^{s} {Γ}^{-} ( \frac{e}{{e}_{s}} ) {ρ}^{Y} - {κ}^{s} s \qquad Eq . ( 9 ) ( sclerostin dynamics )$$

### Resorption Signal

Another component of an in-silico model according to the present teachings for simulating bone mineralization is bone resorption. Bone resorption is a process by which osteoclasts degrade the tissue in bones and release the minerals and signaling factors, such as TGFβ, BMP2, PDGF and IGFs, in the bone matrix. The resorption signal is produced by osteoclasts. In some embodiments of the present teachings, it is assumed that the time scale associated with synthesis and degradation of osteoclasts is much faster than the time scale of osteoclast birth and death so that the following relation holds: r∝ρ^{C}.

More specifically, since the resorption signal acts as a regulator of bone formation and is resealed by threshold concentrations, the proportionality in the above relation can be replaced with equality. In other words, the following relation can be employed: r=ρ^{C}.

### Bone

As noted above and as discussed in more detail below, the present teachings employ the dynamic mathematical relations disclosed herein to simulate bone remodeling. The gain in total bone density is proportional to the osteoblast density, and is downregulated by sclerostin and is upregulated by the resorption signal. Further, the loss in total bone density is proportional to the osteoclast density. In some embodiments of the present teachings, the following dynamic mathematical relations for bone density gain rate (Eq. (10)), the bone density loss (Eq. (11)), the total bone density (Eq. (12)), the bone mineral content (Eq. (13)), and the bone mineral density (Eq. (14)) are employed:

$$\begin{matrix}{b}^{+} = {λ}^{B} {Γ}^{-} ( \frac{s}{{s}_{Ω}} ) [ 1 + {v}^{Ω} {Γ}^{+} ( \frac{r}{{r}_{Ω}} ) ] {ρ}^{B} & Eq . ( 10 ) ( density gain rate ) \\ {b}^{\_} = {λ}^{C} {ρ}^{C} & Eq . ( 11 ) ( density loss rate ) \\ \dot{Ω} = {b}^{+} - {b}^{-} & Eq . ( 12 ) ( total bone density ) \\ \dot{ϕ} = γ [ ξ + {ɛ}_{1} - ( ξ + {ɛ}_{2} ) ϕ ] \\ ϕ = [ 0 , 1 ] , \\ {ɛ}_{1} = 0 , \\ {ɛ}_{2} = 1 & Eq . ( 13 ) ( bone mineral content ) \\ {\dot{Ω}}_{m} = ϕΩ & Eq . ( 14 ) ( bone mineral density )\end{matrix}$$

Although in some embodiments, an exponential dynamics of the form Ω̅=(b⁺−b⁻) Ω may be employed, in the embodiments disclosed herein, a linear variant is employed because (i) Ω is expected to vary only within a range where exponential effects are largely negligible, and (ii) exponential dynamics have the drawback of blowups when the fitting parameters lead to a large ratio

$$\frac{{b}^{+}}{{b}^{-}} .$$

It is also noted that (ϵ₁) and (ϵ₂) are used as dummy parameters that are introduced to provide an interface for model extensions that modulate the mineralization process.

### Overview of Mathematical Model

A model according to an embodiment of the present teachings for simulating the bone mineralization process can be represented mathematically in the following form, which highlights the model's generalizable structure.

In the following S=(s, e, r) is the vector of signaling factor/hormone concentrations and M=(θ^{romo}, θ^{romo}, . . . ) represents a vector ofcompounds that describe time-dependent and time-independent model-external agents such as medications or other forms of treatment, comorbidities, lifestyle-related aspects such as physical activity, diet, et . . . .

The dynamics of precursor populations is given by the following relations:

\dot{ρ}_{C*}=\tilde{μ}_{C*}(s)−f_{C*→C}−\tilde{η}_{C*}(s)ρ_{C*} Eq. (15) (pre-osteoclasts)

\dot{ρ}_{B*}=\tilde{μ}_{B*}(S)−f_{B*→B}−\tilde{η}_{B*}(S)ρ_{B*} Eq. (16) (pre-osteoblasts)

\dot{ρ}_{C}=α_{C*}f_{C*→C}−\tilde{η}_{C}(S)ρ_{C} Eq. (17) (osteoclasts)

\dot{ρ}_{B}=α_{B*}f_{B*→B}−\tilde{η}_{B}(S)ρ_{B}−f_{B→Y} Eq. (18) (osteoblasts)

\dot{ρ}_{Y}=f_{B→Y}−\tilde{η}_{Y}(S)ρ_{Y} Eq. (19) (osteocytes)

\dot{s}=\tilde{μ}ₛ(S)ρ_{Y}−\tilde{κ}ₛ(S)s Eq. (20) (sclerostin)

\dot{Ω}=b⁺−b⁻ Eq. (21) (bone density)

b⁺=\tilde{λ}_{B}(S)ρ_{B} Eq. (22) (bone density)

b⁻=\tilde{λ}_{C}(S)ρ_{C} Eq. (23) (bone density)

ϕ=\tilde{μ}_{ϕ}(S)−\tilde{κ}_{ϕ}(S)ϕ Eq. (24) (bone mineral content)

\dot{Ω}ₘ=ϕΩ Eq. (25) (bone mineral density)

where differentiation rates are of the generic form:

f_{X→Y}=\tilde{ω}_{X}(S)ρ_{X} Eq. (26) (pre-osteoclasts)

For the above incarnation of the above model, the following can hold:

$$\begin{matrix}{\tilde{μ}}_{C *} ( S ) = {μ}_{C *} & Eq . ( 27 ) \\ {\tilde{μ}}_{B *} ( S ) = {μ}_{B *} & Eq . ( 28 ) \\ {\tilde{ω}}_{C *} ( S ) = {Γ}^{-} ( \frac{e}{{e}_{C *}} ) {Γ}^{+} ( \frac{s}{{s}_{C *}} ) {ω}_{C *} & Eq . ( 29 ) \\ {\tilde{ω}}_{B *} ( S ) = {Γ}^{-} ( \frac{s}{{s}_{B *}} ) [ 1 + {v}_{B *} {Γ}^{+} ( \frac{r}{{r}_{B *}} ) ] {ω}_{B *} & Eq . ( 30 ) \\ {\tilde{ω}}_{B} ( S ) = {Γ}^{-} ( \frac{s}{{s}_{Y}} ) {ω}_{B} & Eq . ( 31 ) \\ {\tilde{η}}_{C *} ( S ) = 0 & Eq . ( 32 ) \\ {\tilde{η}}_{B *} ( S ) = 0 & Eq . ( 33 )\end{matrix}$$

### Model Extensions

One advantage of the methods and systems according to the present teachings is that the in-silico models of the present teachings can be readily extended to take into account the effect of a disease and one or more medications on the bone mineralization. By way of example, such extensions can be implemented by keeping the core structure of the model intact. In the following embodiments, an “extension” is defined as a model modification whereby
(i) new dynamic variables may be introduced)
(ii) one or more parameters and/or dynamic variables in the core model may be replaced by a sum (and not a product, power or higher-order operations) of the respective original parameter/variable and one or multiple new terms, whereby
(ii-a) the new terms do not depend on parameters of the core model,
(ii-b) the new terms depend on one or more of the new dynamics variables and auxiliary variables in such a way that the new terms vanish if the dynamic and auxiliary variable are set to zero.

One advantage of the above procedure is that because of (ii) condition, such extensions can be modified without changing the structure of the core model. Further, because of (ii-a) condition, extensions do not change the interpretation of parameters of the core model.

In some embodiments, one or more interfaces can be provided for simulating the effect of a physiological condition (e.g., age, menopause, etc.) a disease and/or a therapeutic treatment on bone remodeling. As discussed in more detail below, in some such embodiments, the therapeutic treatment includes the administration of two or more medications to a patient. Examples of such interfaces are discussed below.

### I. Interface to Model-External Aging-Related Processes

(i) Post-Menopausal Decline of Estrogen Production

It is known that estrogen levels decrease in post-menopausal women. In some embodiments, age-dependent estrogen production in women is simulated and such simulation is employed as an interface to the core model for simulating bone remodeling. More specifically, assuming fast kinetics compared to the change in production rates, the relative estrogen supply (quasi steady-state) can be given by the following relation:

$$e ( a ) = \{ \begin{matrix}1 , a < {a}_{e} \\ \frac{1}{1 + ( a - {a}_{e} ) / {τ}_{e}} , a \geq {a}_{e}\end{matrix} \qquad Eq . ( 34 )$$

where, a_{ϵ} denotes the age at the onset of estrogen decline and τₑ is a characteristic time scale.

### Interface to Biomarkers

### Conventions for Bone Turnover Markers

In the following discussion, to compare the model output to clinical data, the model observables are related to established biomarkers, most notably markers of bone turnover.

Clinical data suggests that the biomarker serum levels change relative to the baseline scales nonlinearly with respect to formation/resorption rate. For instance, serum levels of the bone formation marker PINP (Procollagen type 1 amino-terminal propeptide) peak at about two-fold base levels 3 months after the start of sclerostin mAb treatment but BMD (bone mineral density) changes suggest an approximate 20-fold increase in the bone formation rate.

Hence, in some embodiments, a functional relationship g(b) that relates biomarker concentration M and bone formation/resorption rate τ is assumed to satisfy g (αb₀)=βg(b₀), where b₀ represents the basal rate and the scaling factors α and β describe the change in rate and biomarker concentration after start of treatment, respectively. In particular, in some embodiments, the following relation is employed: g(b)=b^{q}, such that b=(ln β)/(ln(α)).

In some other embodiments, a more flexible approach, which uses two parameters per biomarker, employs the following relation:

φ(b)=c+γb^{q} Eq. (35)

where c represents basal biomarker levels and γ paramterizes the dependence of the biomarker on the respective turnover rate.

By way of example, in some embodiments, the following bone turnover markers can be employed:

(i) Bone Formation:
Bone-specific alkaline phosphate (HSAP),
Procollagen type 1 amino-terminal propeptide (PINP)

(ii) Bone resorption:
C-terminal telopeptide (CTX)

In this embodiment, the following relations are employed for the following biomarkers: BSAP, PINP, and CTX

φ_{BSAP}(b⁺)=(b⁺)^{qBSAP} Eq. (36)

φ_{PINP}(b⁺)=(b⁺)^{qPINP} Eq. (37)

φ_{CTX}(b⁻)=(b⁻)^{qCTX} Eq. (38)

The above relations indicate that the concentrations of biomarkers BSAP and PINP depend on the bone formation rate b⁺, whereas concentration of the biomarker CTX depends on the bone resorption rate b⁻. Further, the relations indicate that three relations include different exponents.

### II. Model Extensions

In some embodiments, an ire-silica model according to the present teachings can provide modules for determining the effects of one or more medications on the progression of osteoporosis. In particular, in some such embodiments, such modules can be configured to determine the effect of the virtual administration of a combination of two or more medications on the progression of osteoporosis. In particular, an in-silico model according to the present teachings can take into account the cross reactions of two or more medications via capturing their mechanisms of action and determine their joint downstream effects relevant for bone turnover.

### Pharmacokinetics

In some embodiments, the presence of a medication in a patient's body can be represented by an effective (dimensionless) variable 0 that indicates the relative concentration of a medication. Typically, the variable 0 is given in multiples of some threshold that parametrizes the effect of the drug. In general, the pharmacokinetics of a particular medication can inform the interpretation of 0.

In many embodiments, the pharmacokinetics of any drug X can be described by the following two parameters: the efficacy E_{X} and the medication's half-life T_{X}. A variety of different pharmacokinetic models can be employed. For example, for repeated administrations with doses c₁, . . . , cₙ at times t₁, . . . , tₙ, the relative concentration variable for drug X can be given by the following relation:

θ^{X}(t)=E_{X}Σᵢ₌₁ⁿcᵢ2^{-(t-t}^{ᵢ}^{)/T}^{_{X}}Θ(t−tᵢ) Eq. (39)

where Θ is the Heaviside step function. It should be understood that other pharmacokinetic models can also be utilized in the practice of the present teachings. For example, the above relation (39) can be replaced with other relations indicative of different pharmacokinetic models.

By way of example, the present teachings can be employed to determine the effect of RANKL inhibitors, Sclerostin antibodies, and bisphosphonates on the progression of osteoporosis as discussed in more detail below. It should, however, be understood that the present teachings are not limited to these medications, but rather can be employed to model the effects of other medications, as well as combinations of two or more medications, on bone remodeling, and in particular the progression of osteoporosis.

### RANKL Inhibitors

A RANKL inhibitor can bind the RANKL and block its interactions with RANK, which, in turn, affects pre-osteoclast differentiation.

Denosumab, a monoclonal antibody, is an example of a RANKL inhibitor that binds with affinity to RANKL. Further, without being limited to a particular theory, Denosumab appears to facilitate the secondary mineralization, which leads not only to a halt in BMD decline but also to a long-term increase in BMD. Without being limited to a particular theory, Denosumab suppresses osteocytic dissolution action, which can allow for progression of the secondary mineralization process and an increase in overall BMD.

In some embodiments, the extension of an in-silico model according to the present teachings for taking into account the effect of virtual administration of a RANKL inhibitor can be achieved by providing an interface to the variables indicating the differentiation rate of pre-osteoclasts to osteoclasts and mineralization. More specifically, these variables can be adjusted as shown below by the use of additional regulatory elements:

ω^{C*}→ω^{C*}−ωᵣ₋ᵢₙₕ^{C*}Γ⁺(θ^{r-inh}) Eq. (40)

ϵ→ϵ−υᵣ₋ᵢₙₕ^{φ}Γ⁺(σ^{r-inh}) Eq. (41)

where θ^{r-inh} is the functional RANKL inhibitor concentration in multiples of EC₅₀, which, for simplicity, in this embodiment, it is assumed to be identical for the regulation of differentiation and mineralization. Furthermore, ωᵣ₋ᵢₙₕ^{C*}<ω^{C*} and νᵣ₋ᵢₙₕ^{φ} parametrize the respective maximum effect strength.

### Sclerostin Antibodies

In some embodiments, the present teachings provide an interface to one or more variables that allow simulating the effect of the administration of sclerostin antibody to an individual. More specifically, in some embodiments, the effects of the administration of a sclerostin antibody can be taken into account by modifying the sclerostin dynamics by an additional regulatory element via the replacement \dot{s} of \dot{s}* and variables as follows:

\dot{s}→\dot{s}−κ_{mAb}^{s}θ^{mAb}s+δ^{s}s* Eq. (42)

\dot{s}*=κ_{mAb}^{s}θ^{mAb}s−κ_{*}^{s}s*−δ^{s}s* Eq. (43)

Where θ^{mAb} is the functional antibody concentration and κ^{s} is the sclerostin degradation rate; this parametrization implies that θ^{mAb} is given in multiples of the antibody concentration needed to achieve the same binding rate as the natural degradation process of sclerostin. Furthermore, s* is the concentration of antibody-bound sclerostin with ds being the unbinding rate.

To provide a single interface for multiple sclerostin antibodies, one can define the effective Romosozumab and Blosozumab doses θ^{mAb}=E^{X}\ddot{θ}^{X}, where θ^{X} is the absolute antibody dose and the efficacy E^{X} rescales \ddot{θ}^{X} to a dimensionless number that represents its relative contribution to the total effects of the antibodies. Thus,

θ^{mAb}=θ^{romo}+θ^{bloso} Eq. (44)

### Bisphosphonates

Bisphosphonates (e.g., Alendronate, Risedronate, Ibandronate, Zoledronate) inhibit osteoclast-mediated bone resorption by promoting osteoclast apoptosis.

In some embodiments, the effect of the administration of bisphosphonates on bone mineralization, and in particular, on the progression of osteoporosis can be modeled by providing an interface for adjusting the osteoclast apoptosis rate as follows:

\dot{ρ}^{C}→\dot{ρ}^{C}−η_{bisph}^{C}Γ⁺(θ^{bisph})ρ^{C} Eq. (43)

where η_{bisph}^{C} is the reference additional apoptosis rate due to the presence of bisphosphonates.

In some embodiments, in order to provide a single interface for multiple bisphosphonates, effective doses θ^{X}=E^{X}\ddot{θ}^{X} for X=alendronate, ibandronate, zoledronate, . . . , where \ddot{θ}^{X} is the absolute bisphosphonate dose and the efficacy E^{X} rescales \ddot{θ}^{X} to a dimensionless number that represents its relative contribution to the total effects of antibodies. Thus,

θ^{bisph}=θ^{alendro}+θ^{ibandro}+θ^{zoledro}+ . . . Eq. (46)

### Model Extensions for Causes of Secondary Osteoporosis

In some embodiments, a model according to the present teachings can be configured to simulate the effect of a disease on bone mineralization, and particularly, on the progression of osteoporosis. For example, in some embodiments, the present teachings can provide an interface to one or more variables that can be adjusted to account for the effects of a disease on bone mineralization.

Some examples of diseases for which the present teachings can provide an interface to the core in-silico model for simulating their effects on bone mineralization are discussed below. It should, however, be understood that the present teachings can be applied to simulate the effects of other diseases on bone mineralization as well, e.g., by providing an interface to one or more dynamic variable of the core model.

### Multiple Myeloma

Multiple myeloma (MM) is a B-cell malignancy characterized by the clonal proliferation of malignant plasma cells in the bone marrow microenvironment. In MM, bone resorption is stimulated and new bone formation is inhibited. In particular, MM cell can stimulate osteoclast differentiation via production of IL-3, DcR3, CCL3, MIP-1β, VEGF, TNF-α, and RANKL. Further, MM cells can also adhere to bone marrow stromal cells via very late antigen 4 (VLA-4) and vascular cell adhesion molecule 1 (VCAM-1), which can in turn promote osteoclast differentiation and activation.

In this embodiment, the pre-osteoclast to osteoclast differentiation can be employed as an interface to the variable indicative of the pre-osteoclast to osteoblast differentiation rate (ω^{C}) in the following way:

$${ω}^{C} \to {ω}^{C} [ 1 + {β}_{mm}^{C} ] \tilde{Θ} ( \frac{t - {t}_{mm}}{{τ}_{mm}} ) \qquad Eq . ( 47 )$$

where tₘₘ is the time of onset, τₘₘ is the duration of the build-up and \tilde{Θ} is a smooth step function.

### Glucocorticoid-Induced Osteoporosis

Glucocorticoids are corticosteroids, which are a class of hormones that are produced by the adrenal gland. Glucocorticoids have a role in regulating the glucose metabolism but can also modulate the immune system, where they can suppress the immune function.

Glucocorticoids can be used to treat over-functions of the immune system, such as allergies, asthma, autoimmune diseases, and after transplantation.

Glucocorticoid-induced osteoporosis (GIOP) is known to be the most common cause of secondary osteoporosis. High levels of glucocorticoids can dramatically decrease bone formation rate, osteoblast numbers and osteocyte numbers and activity.

Glucocorticoids can also increase the production of macrophage colony stimulating factor (M-CSF) and RANKL, and decrease the production of osteoprotegerin (OPG) by osteoblastic cells and osteocytes, thus resulting in an increase in both the number and activity of osteoclasts.

Glucocorticoid-induced osteoporosis can result in a rapid bone loss due to a combination of increased bone turnover and an inhibition of bone formation.

In some embodiments, the present teachings employ the reference gain rates and the differentiation rates associated with osteoblasts, osteocytes, and pre-osteoclasts as interfaces for simulating the effects of glucocorticoids on bone mineralization, and in particular their effects or the progression of osteoporosis. For example, in this embodiment, these gain and differentiation rates can be adjusted as indicated below to simulate the effects of glucocorticoids on osteoporosis:

$$\begin{matrix}{η}^{B} \to {η}^{B} [ 1 + {v}_{gc}^{B} {Γ}^{+} ( \frac{{θ}^{gc}}{{θ}_{B}^{gc}} ) ] & Eq . ( 48 ) \\ {η}^{Y} \to {η}^{Y} [ 1 + {v}_{gc}^{Y} {Γ}^{+} ( \frac{{θ}^{gc}}{{θ}_{Y}^{gc}} ) ] & Eq . ( 49 ) \\ {ω}^{C *} \to {ω}^{C *} [ 1 + {v}_{gc}^{C *} {Γ}^{+} ( \frac{{θ}^{gc}}{{θ}_{C *}^{gc}} ) ] & Eq . ( 50 ) \\ {α}^{C *} \to {α}^{C *} [ 1 + {v}_{gc}^{C} {Γ}^{+} ( \frac{{θ}^{gc}}{{θ}_{C}^{gc}} ) ] & Eq . ( 51 )\end{matrix}$$

As discussed below, in some embodiments, an in-silico model according to the present teachings can be used to implement a “Virtual Clinic.” For example, an in-silico model according to the present teachings can be used to predict the progression of osteoporosis based on data gathered from an individual patient or a collection of patients. In such an implementation, avatars (herein also referred to “digital twins”) are generated as representations of individual patients or patient populations through a set of patient-specific parameters that characterize their physiological response within the scope of a physiological mathematical model according to the present teachings.

By way of example, avatars can be used to predict patient-individual response and to optimize treatments on a personalized basis. In some embodiment, a large number of avatars, e.g., in a range of 1 to a few thousands, for example, more than 10,000, can be used to optimize general population level treatment schemes in a “Virtual Trial” setting so as to find a treatment (or a set of treatments) that collectively maximize the desired clinical outcome.

By way of example, in some embodiments, within an in-silica model according to the present teachings, avatars can be defined as follows. In particular, in many embodiments, each parameter associated with the model and its extensions can fall into the following two categories:

(1.) Parameters that are approximately constant among a patient population of interest,

(2) Parameters that reflect patient-specific physiological responses and properties.

An avatar (which is also herein referred to as a digital twin) is defined as a specific set of values for parameters in the second category. Such parameters may include, for example, the osteoporosis-related physiology of an individual patient or a patient population with similar characteristics.

For a specific patient population, an avatar may be found by fitting the model to clinical datasets (e.g., to hone mineral density measurements or measurements of biomarkers) and/or by inferring parameters from model-external properties (such as age, gender and other demographic properties). By way of example, the following datasets may be used to create avatars:

### Dynamic Data

Bone mineral density (BMD), e.g., through DXA, X-ray, and/or quantitative CT.
Bone turnover markers
   i) PINP serum concentration
   ii) BSAP serum concentration
   iii) β-CTX serum concentration
Lab measurements
   i) Estrogen serum concentration
   ii) Sclerostin serum concentration
a Drug administration data (doses, administration dates)
   i) Sclerostin antibodies (Romosozumab, Blosozumab, etc.)
   ii) RANKL inhibitors (Denosumab etc.)
   iii) Bisphosphonates (Alendronate, Risedronate, Ibandronate, Zoledronate, etc.)

### Static Data

Demographics
   i) Age
   ii) Gender
   iii) Race
   iv) Ethnicity
   v) Height
   vi) Weight

In some embodiments, the model can be based on data from individual patients. Such an approach would typically require a long history of measurements (e.g., many years, or even decades) for each individual patient. In some cases, it may be possible to infer patient-specific parameters with high confidence from auxiliary data.

### Physical Activity and Altered Gravity

In some embodiments, an in-silico model according the present teachings allows simulating the effect of a physical activity and/or altered gravitational conditions in bone remodeling, and in particular in treatment and/or progression of osteoporosis.

For example, in a model according to an embodiment, the effects of a time-dependent variation in physical activity can be incorporated by a dynamical inhibition of sclerostin. Let ϑₓ (t) denote a coarse-grained normalized physical activity, e.g., fraction of a month spent on exercises of type x (e.g., running, cycling, weight lifting), such that ϑₓ=1 corresponds to the average of a considered patient population. A corresponding module modulates the sclerostin production according to

μ^{s}→μ^{s}+2μ_{phys}^{s}(Γ⁻(ϑₓ)−½) Eq.(52)

where μ_{phys}^{s<μ}^{^{s}}^{.}

Similarly, the effects of altered gravitational conditions, e.g., higher or lower gravity, can be incorporated into an in-silico model according to the present teachings. For example, a relation similar to the above Eq. (52) can be employed in which another variable ϑₓ(t) with type x indicating gravity as an effect can be employed, so that ϑₓ=1 corresponds to normal conditions (i.e., gravity on the earth's surface), ϑₓ<1 corresponds to lower gravity and ϑₓ>1 corresponds to higher gravity.

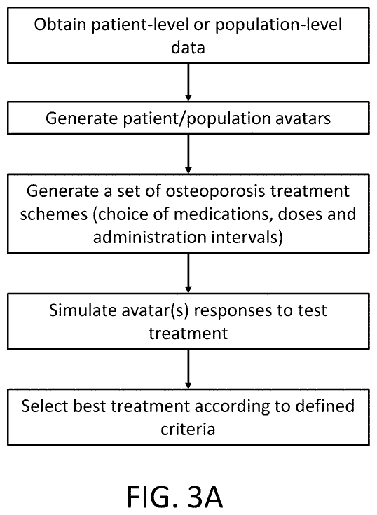

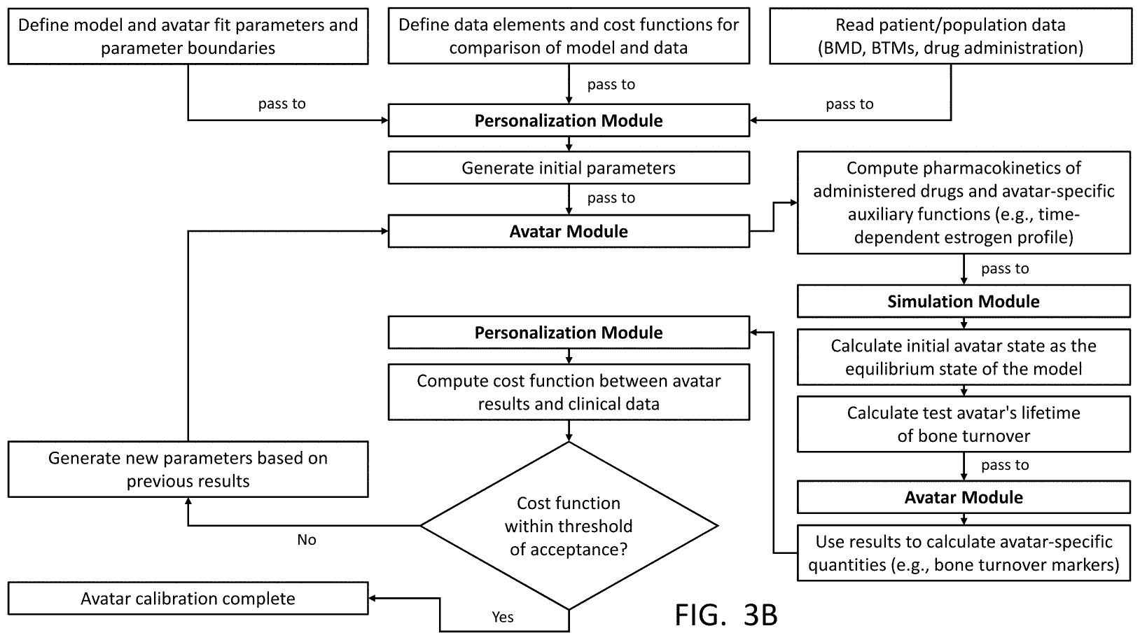

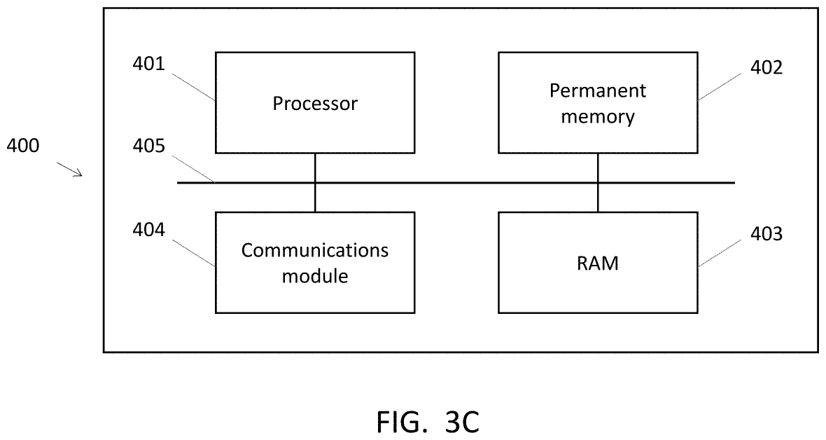

In some embodiments, the present teachings allow the selection of an optimal protocol for treatment of osteoporosis (e.g., slowing the progression rate of osteoporosis). By way of example, such selection of an optimal protocol can include the selection of an optimal dosage and/or administration regimen of a drug. With reference to FIG. 3A, in one such embodiment, patient-level or population level clinical data are employed to generate patient-specific or population avatars. A set of osteoporosis treatment schemes (e.g., such as the choice of medications, doses, and administration intervals) can be defined. Avatar responses to the set of treatment schemes can be simulated based on the present teachings, and an optimal treatment scheme can be selected based on the results of the simulations.

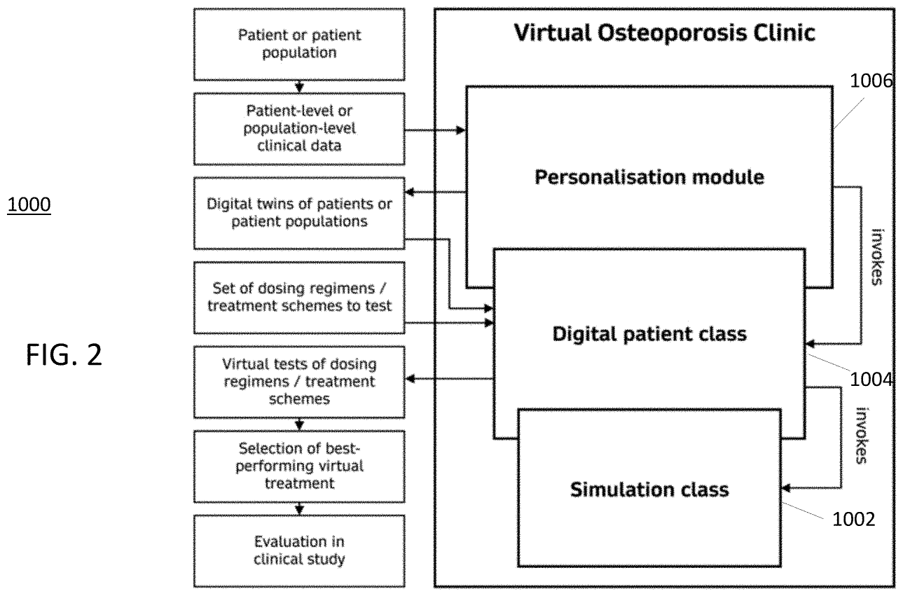

FIG. 2 schematically depicts an example of a system 1000 according to an embodiment (herein also referred to as a virtual osteoporosis clinic) for implementing an in-silico model according to the present teachings. The system 1000 includes a simulation module 1002, a digital patient module (herein also referred to as an avatar module) 1004, and a fitting module 1006 (which is herein also referred to as a personalization module). As in this embodiment the simulation module, the avatar module and the fitting module are implemented as classes in an object-oriented programming environment, they are also referred to herein as the simulation class, the avatar class and the fitting class.

As discussed below, these modules are in communication with one another to provide in-silico simulation of bone remodeling, and in particular, progression of osteoporosis using the dynamic mathematical relations discussed above. In particular, FIG. 3B provides a workflow diagram that shows the flow of information between these modules.

With reference to both FIGS. 2 and 3B, in this embodiment, the fitting module 1006 (which is also herein referred to as a personalization module) receives patient-level or population-level data. By way of example, such data can include bone density and biomarker serum measurements over time (e.g., time-series of bone density and biomarker serum measurements), treatment-related data, such as doses and time points of drug administration, as well as static patient data, such as initial age, onset of menopause, etc.). The fitting module 1006 is also provided with defined data elements and cost functions or comparison of model and data as well as defined model and avatar fit parameters and parameter bounds.

By way of example, and without limitation, a cost function may be defined as a weighted sum of residuals. For example, let Ω_{m,i} and Ω̅_{m,i} denote clinical measurements and simulation results, respectively, for the bone mineral density Ωₘ at time points tᵢ with i=1, . . . n. The measured and simulated serum concentrations of P1NP (φᵢ^{P1NP} and φ̅ᵢ^{P1NP}) and CTX (φᵢ^{CTX} and φ̅ᵢ^{CTX}) can be defined in an analogous way. The simulated values are functions of the set of model parameters P. where the sum of squared differences is defined as follows:

D₁(P)=Σᵢ₌₁ⁿ(Ω_{m,i}−Ω̅_{m,i}(P))² Eq. (53)

D₂(P)=Σᵢ₌₁ⁿ(φᵢ^{P1NP}=φ̅ᵢ^{P1NP}(P))² Eq. (54)

D₃(P)=Σᵢ₌₁ⁿ(φᵢ^{CTX}=φ̅ᵢ^{CTX}(P))²Eq . (55)

Then, one possible cost function is given by

C(P)=Σ_{κ=1}³w_{κ}D_{κ}(P) Eq. (56)

where the wᵢ denote the weights of the respective quantity in the cost function.
An alternative cost function can be defined as a sum of logarithms, which renders the cost function less dependent on the absolute scale of respective quantities,

C(P)=Σ=κ_{κ=1}³wₖ ln D_{κ}(P) Eq. (57)

Other cost functions can also be employed in the practice of the present teachings.

The fitting module is configured to fit the data to a predefined model so as to derive parameters that will be employed by the avatar module 1004. Some examples of model parameters include, without limitation, thresholds for regulation of bone turnover through estrogen, sclerostin; rates of cell birth, differentiation and death; rates of bone formation and resorption per cell density. Typical avatar parameters can include efficacy and half-life of the administered drugs, parameters determining the relation between bone turnover and bone turnover markers found in a patient, time scale of estrogen decline after onset of menopause, etc. The fitting module then formats the data so as to enable its comparison with simulations and sets up a parameter fit strategy.

To fit the parameters, the fitting module 1006 invokes the avatar module to compute thousands of avatar realizations and compares them with the data. For each realization, the fitting module 1006 passes a candidate parameter set (i.e., avatar and model parameters) to the avatar module and the avatar module computes the entire lifetime of bone evolution for that avatar.

For example, as shown in the workflow chart of FIG. 3B, the fitting module 1006 can generate an initial set of parameters and pass that initial set of parameters to the avatar module. The avatar module 1004, in turn, computes all patient-specific auxiliary variables such as the age-dependent estrogen level and the initial conditions. For example, the avatar module 1004 computes the pharmacokinetic profile of administered drugs and avatar-specific auxiliary functions, such as the time-dependent estrogen profile.

Subsequently, the avatar module 1004 passes this information, together with the entire set of parameters, to the simulation module 1002, which employs a numerical solver to compute a solution, of the bone remodeling (osteoporosis) model according to the present teachings using all parameters and auxiliary functions obtained from the avatar module 1004. In particular, as depicted in the workflow of FIG. 3B, the simulation module computes the initial avatar state as the equilibrium state of the model and calculates test avatar's lifetime of bone turnover.

The simulation module 1002 then passes this information back to the avatar module 1004. The avatar module 1004 employs this information to compute additional avatar-specific quantities, such as time-dependence of the bone turnover markers. In general, in many embodiments, the avatar module handles all the characteristic that are specific to the patient.

The avatar module 1004 then compiles all the results and passes the compiled information back to the fitting module 1006. The fitting module 1006 in turn computes the value of a cost function, which had been passed earlier to the fitting module as discussed above, between the avatar results and the clinical data. If the calculated cost function, or a reduction in calculated value of the cost function, is below an acceptable threshold, the avatar calibration is completed. However, if the calculated cost function, or a reduction in calculated value of the cost function, is not below acceptable threshold, the fitting module 1006 will generate new parameter set based on the previous results, and passes the new parameters to the avatar module. This process is repeated iteratively until an acceptable convergence of the model to the clinical data is achieved. In some cases, it is possible for the value of the best (i.e., the optimal) cost function to be greater than a preset threshold. In such a situation, the avatar associated with that cost function can be rejected. In this embodiment, the fit parameters vary between different candidate sets while all other parameters remain fixed to a reference value.

The workflow for obtaining an optimal virtual treatment using an osteoporosis model according to the present teachings can vary depending on the type of scenario under investigation. For example, in some cases, it may be desired to optimize a dosing scheme for a single drug. In such a case, there are different candidate dosing schemes (e.g., administering the same total drug dose but changing the dosage and the intervals between the administration of the drug). The system 1000 can be used to run each scheme for a population-level avatar or for an entire set of different patient-level avatars (i.e., a so-called Virtual Trial). Population-level avatars can be generated, for example, based on averages of parameters associated with members of a population. The probed dosing schemes can be ranked in accordance with their performance. For instance, in some cases, the desired goal may be the maximization of gain in bone mineral density, or the desired goal may be to optimize the temporal period after a treatment during which the bone density gain due to the treatment is sustained, or combinations thereof.

In some cases, the system 1000 can be employed to probe combination therapies. The workflow is similar to that discussed above, where certain dosing schemes (e.g., sequential and parallel administration of different drugs in different relative doses/intervals, etc.) can be probed using the generated avatars and ranked according to a pre-defined performance criterion. By way of example, such criteria may be based on maximizing the time until BMD falls below the baseline level at the start of the treatment, and/or minimizing potential side effects of a drug. In some embodiments, the criteria can be based on maximizing the total BMD gain (e.g., maximum BMD at some point during the treatment). In some embodiments, each of the simulation module 1002, the avatar module 1004, and the fitting module 1006 can be implemented in software, where each module is implemented as a software layer. For example, in some implementation, an object-oriented programming language, such as Java® or Python®, can be employed and each of the simulation module 1002, the avatar module 1004, and the fitting module 1006 can be implemented as a class, which can be instantiated as an object during execution.

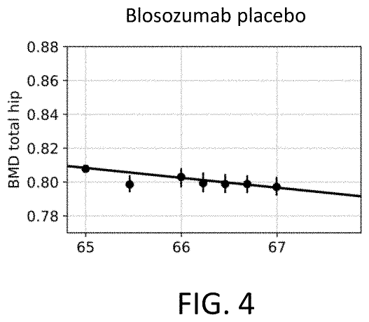

The software modules can be executed on any suitable hardware platform. By way of example, FIG. 4 schematically depicts such a hardware platform 400 that includes, among other components, a processor 401, a permanent memory 402, a random access memory (RAM) 403, and a communications module (WIFI or Bluetooth) 404, and a communications bus 405 for connecting the processor 400 to these components.

By way of example, the processor 400 can be a general and/or special purpose microprocessor, such as an application-specific instruction set processor, graphics processing unit, physics processing unit, digital signal processor, image processor, coprocessor, floating-point processor, network processor, and/or any other suitable processor that can be used in a digital computing circuitry. Alternatively or additionally, the processor can comprise at least one a multi-core processor and a front-end processor. By way of example, the permanent memory 402 can be, for example, magnetic disks (e.g., internal or removable disks), magneto-optical disks, one or more of a semiconductor memory device (e.g., EPROM or EEPROM), flash memory, CD-ROM, and/or DVD-ROM disks.

Instructions and data for performing in-silico simulations according to the present teachings can be stored in the permanent memory and can be transferred onto the RAM during execution.

The following examples are provided for further elucidation of various aspects of the present teachings and are not intended to necessarily indicate the optimal ways of practicing the present teachings and/or the optimal results that may be obtained.

### Examples

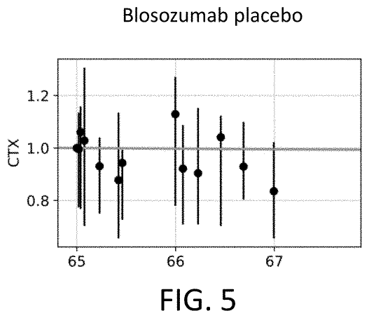

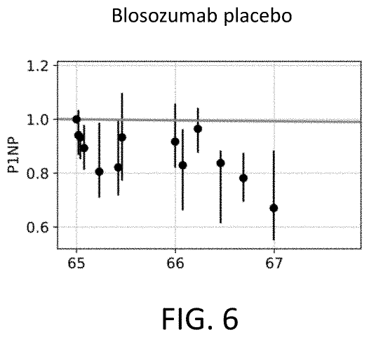

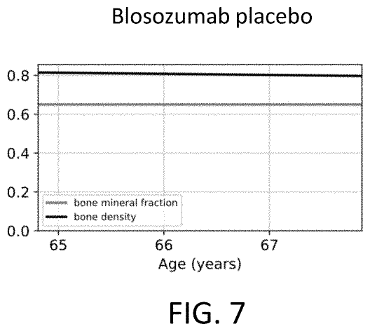

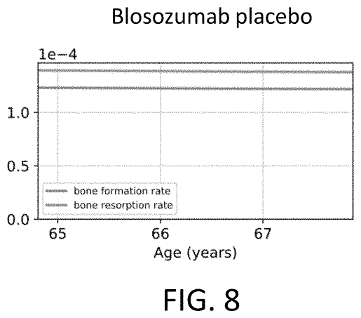

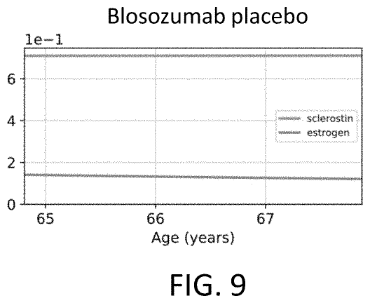

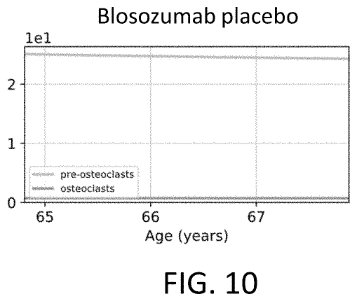

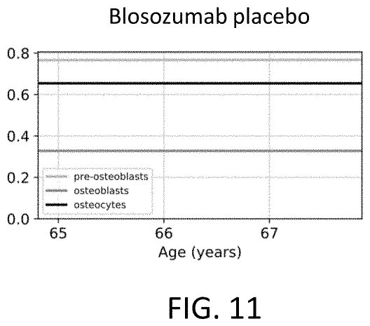

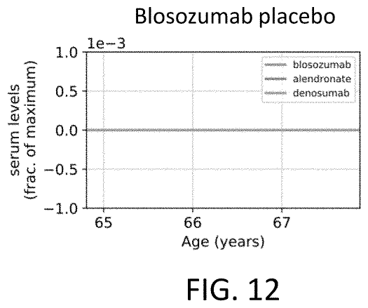

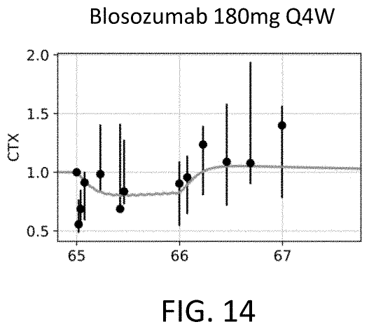

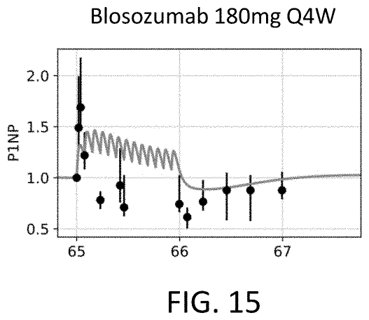

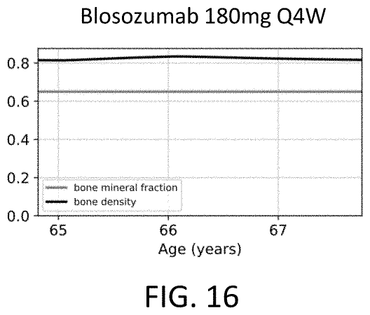

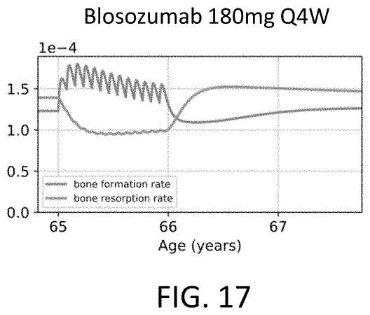

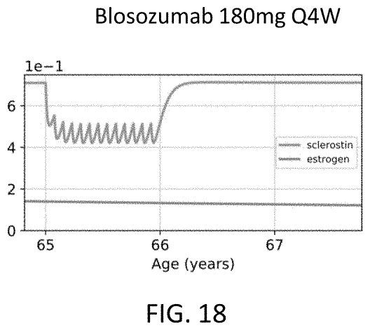

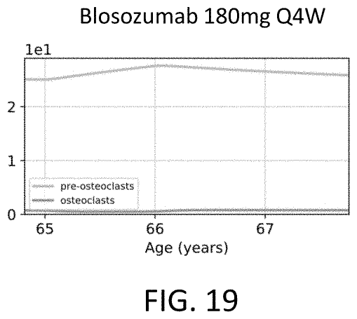

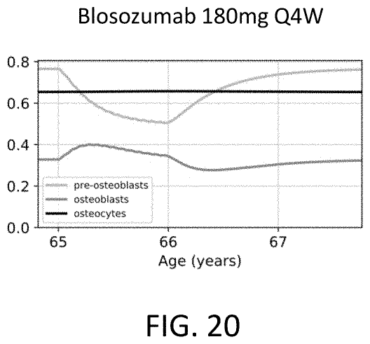

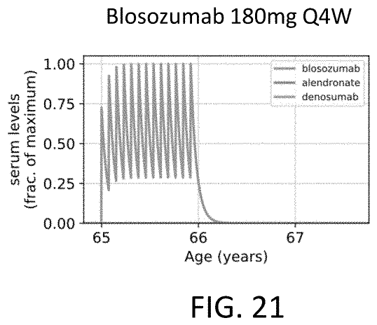

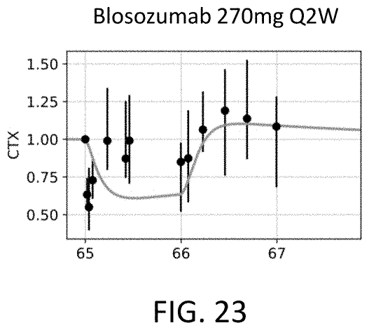

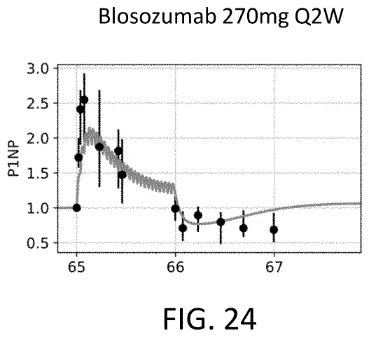

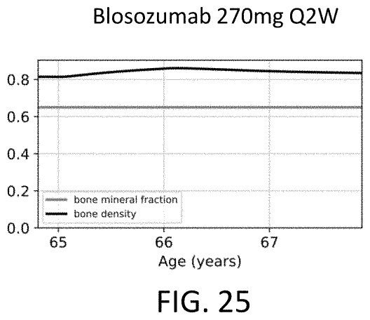

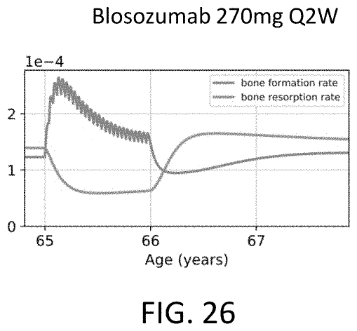

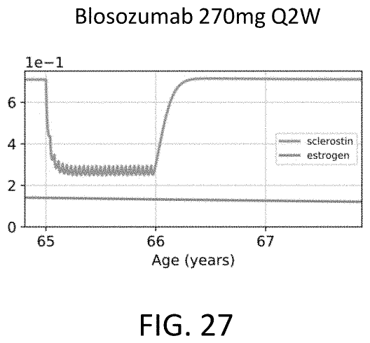

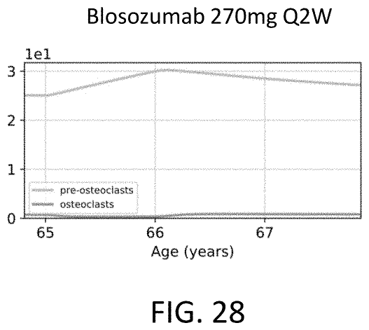

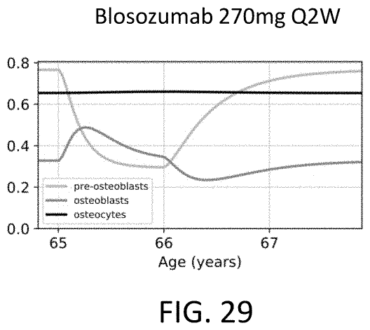

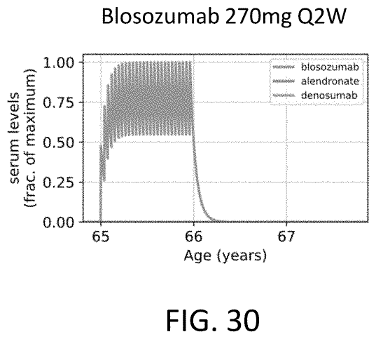

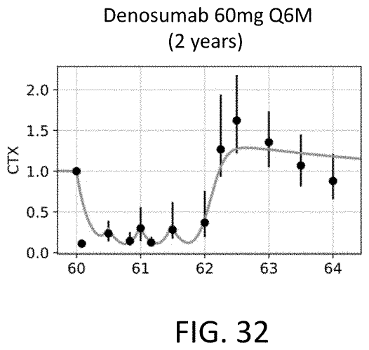

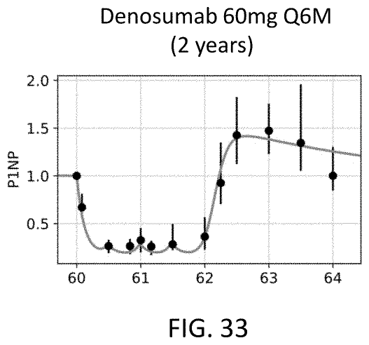

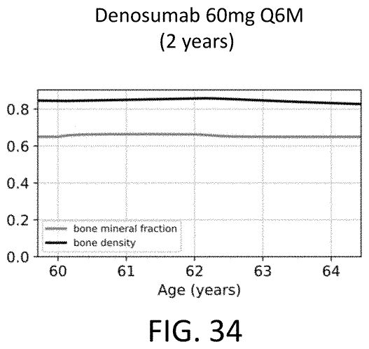

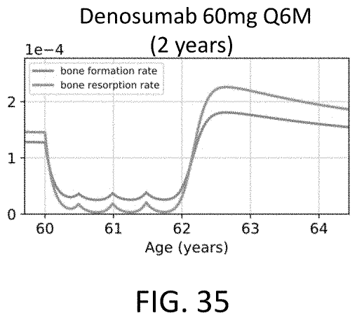

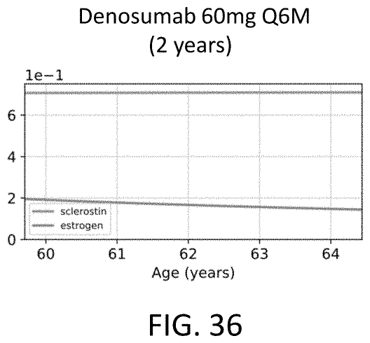

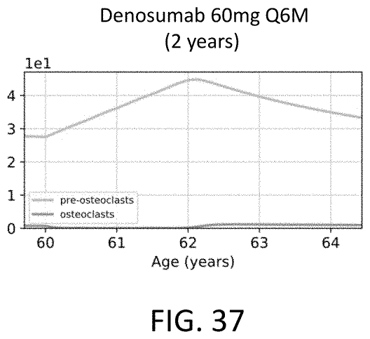

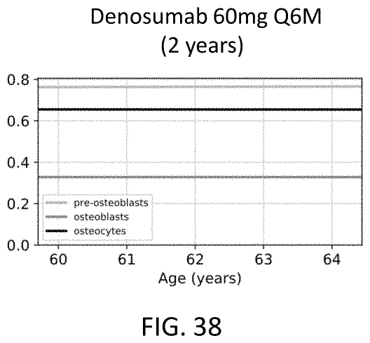

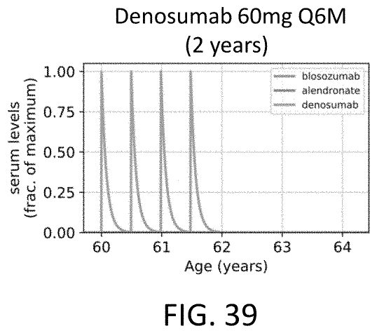

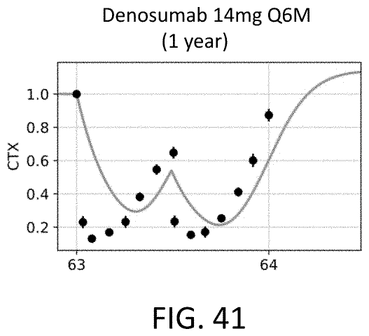

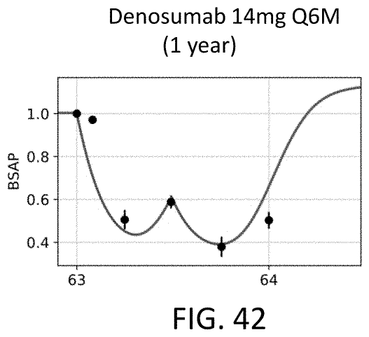

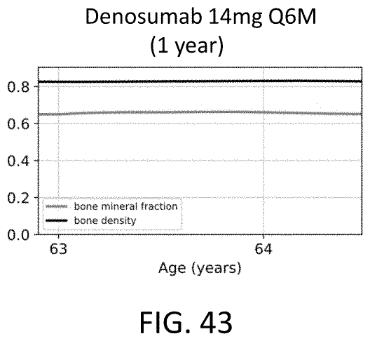

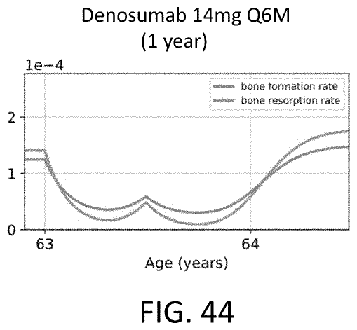

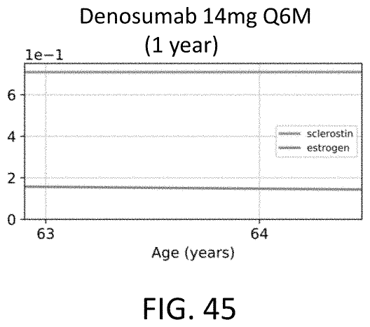

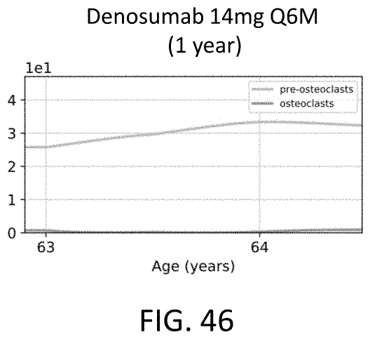

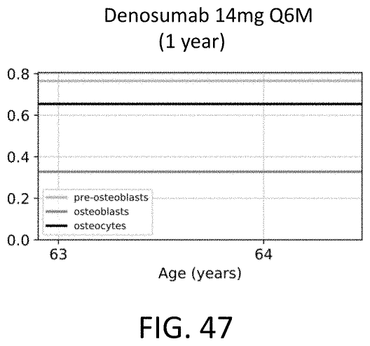

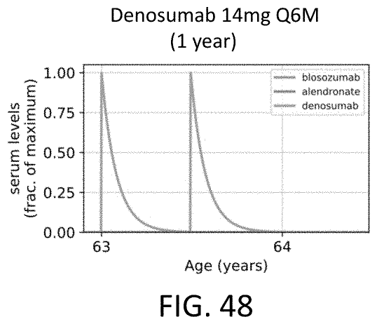

An example of an implementation of methods and systems according to the present teachings for in-silico simulation of osteoporosis was employed to obtain the simulation results depicted in FIGS. 4-48. In these graphs, dots indicate clinical data and curves indicate simulation results.

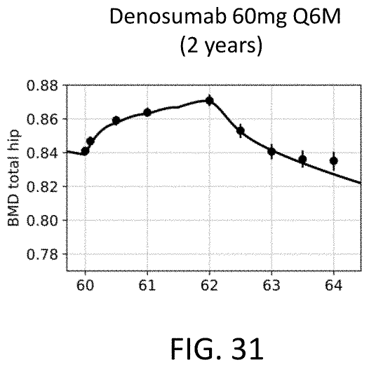

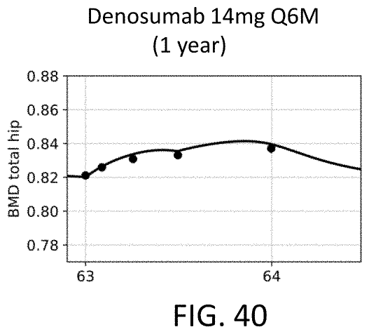

Parameters of the osteoporosis model were obtained using a simultaneous numerical fit of the model including modules for sclerostin antibodies and RANKL inhibitors to clinical datasets. The following clinical datasets were employed for obtaining the depicted simulation results: Recknor et al., J. Bone Miner. Res. 30 (9), 1717-1725 (2015) (FIGS. 4-6, 13-15, 22-24); Bone et al., J. Clin. Endocrinol. Metab. 96, 972 (2011) (FIGS. 31-33), and McClung et al., New Engl. J. Med. 354 (8), 821 (2006) (FIGS. 40-42).

A fit of the model to the clinical data performed in a manner discussed above resulted in the following parameter list:

Parameter list

Value

Model parameters

| | |
|---|---|
| α_{B*} | 0.82 |
| α_{C*} | 0.68 |
| δₛ | 0.05 |
| e_{B} | 94 |
| e_{C} | 1 |
| e_{C*} | 1 |
| eₛ | 10 |
| η_{B} | 0.012 |
| η_{C} | 0.024 |
| η_{Y} | 0.00011 |
| kₑ | 0.64 |
| κₛ | 0.050 |
| λ_{B} | 0.00050 |
| λ_{C} | 0.00019 |
| μ_{B*} | 0.0048 |
| μ_{C*} | 0.025 |
| μₛ | 0.054 |
| n | 1 |
| ν_{C} | 0.2 |
| ν_{Ω} | 88 |
| ν_{B*} | 0 |
| ω_{B} | 0.00022 |
| ω_{B*} | 0.45 |
| ω_{C*} | 0.047 |
| ϕ₁ | 1.86 |
| r_{C} | 1 |
| r_{Ω} | 14 |
| r_{B*} | 1.71 |
| s_{Ω} | 0.12 |
| s_{Y} | 89 |
| s_{B*} | 0.01 |
| s_{C*} | 28 |
| σ | 0.0047 |

Virtual patient parameters

| | |
|---|---|
| E_{blosozumab} | 0.0082 |
| T_{blosozumab} | 15 |
| E_{denosumab} | 26 |
| T_{denosumab} | 21 |
| αₑ | 49 |
| τₑ | 2.6 |
| ωᵣ₋ᵢₙₕ^{C*} | 1 |
| νᵣ₋ᵢₙₕ^{ϕ} | 0.064 |
| q_{BSAP} | 0.67 |
| q_{P1NP} | 1 |
| q_{CTX} | 0.57 |

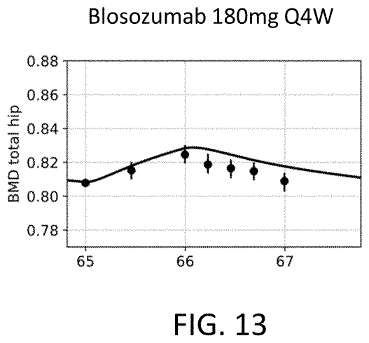

FIGS. 4-12 show graphs indicating simulated changes, as a function of age, in BMD (normalized to first available data point), CTX (normalized to first available data point), PINP (normalized to first available data point) and bone mineral density (arbitrary units), bone formation and resorption rate (in units of maximum bone density levels per day), sclerostin and estrogen (arbitrary units), pre-osteoclasts and osteoclasts (in mutually different arbitrary units), and pre-osteoblasts, osteoblasts, and osteocytes cell population levels (in mutually different arbitrary units) for administration of placebo rather than Blosozumab (i.e., for zero serum levels of Blosozumab as shown in FIG. 13).

FIGS. 13-21 show graphs indicating simulated changes, as a function of age, in BMD (normalized to first available data point), CTX (normalized to first available data point), PINP (normalized to first available data point), bone mineral fraction and bone mineral density (arbitrary units), bone formation and resorption rate (in units of maximum bone density levels per day), sclerostin and estrogen (arbitrary units), pre-osteoclasts and osteoclasts (in mutually different arbitrary units), and pre-osteoblasts, osteoblasts, and osteocytes cell population levels (in mutually different arbitrary units) and serum levels of Blosozumab (relative to maximum level) for administration of 180 mg of Blosozumab every four weeks for one year.

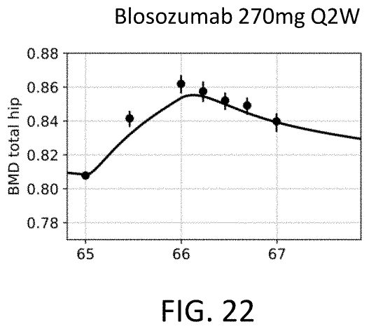

FIGS. 22-30 show graphs indicating simulated changes, as a function of age, in BMD (normalized to first available data point), CTX (normalized to first available data point), PINP (normalized to first available data point), bone mineral fraction and bone mineral density (arbitrary units), bone formation and resorption rate (in units of maximum bone density levels per day), sclerostin and estrogen (arbitrary units), pre-osteoclasts and osteoclasts (in mutually different arbitrary units), and pre-osteoblasts, osteoblasts, and osteocytes cell population levels (in mutually different arbitrary units) and serum levels of Blosozumab (relative to maximum level) for administration of 270 mg of Blosozumab every two weeks for one year.

FIGS. 31-39 show graphs indicating simulated changes, as a function of age, in BMD (normalized to first available data point), CTX (normalized to first available data point), PINP (normalized to first available data point), bone mineral fraction and bone mineral density (arbitrary units), bone formation and resorption rate (in units of maximum bone density levels per day), sclerostin and estrogen (arbitrary units), pre-osteoclasts and osteoclasts (in mutually different arbitrary units), and pre-osteoblasts, osteoblasts, and osteocytes cell population levels (in mutually different arbitrary units) and serum levels of Denosumab (relative to maximum level) for administration of 60 mg of Denosumab every six months for two years.

FIGS. 40-48 show graphs indicating simulated changes, as a function of age, in BMD (normalized to first available data point), CTX (normalized to first available data point), BSAP (normalized to first available data point), bone mineral fraction and bone mineral density (arbitrary units), bone formation and resorption rate (in units of maximum bone density levels per day), sclerostin and estrogen (arbitrary units), pre-osteoclasts and osteoclasts (in mutually different arbitrary units), and pre-osteoblasts, osteoblasts, and osteocytes cell population levels (in mutually different arbitrary units) and serum levels of Denosumab (relative to maximum level) for administration of 14 mg of Denosumab every six months for one year.

Those having ordinary skill in the art will appreciate that various changes can be made to the above embodiments without departing from the scope of the invention.

### APPENDIX I

The transient population burst through a step-like input can be computed as discussed below.

Consider a progenitor population x and an active/mature population y, subject to the dynamics

\dot{x}=μ−ωϕ(t)x

\dot{y}=ωϕ(t)x=κy

where ϕ is an external input. Initially, ϕ=1 and x and y are at steady state, x₀=μ/ω and y₀=μ/κ. At time t=0, ϕ jumps to a new value ϕ₀>1, so that

$$x ( t ) = \frac{{x}_{0}}{{ϕ}_{0}} ( 1 + ( {ϕ}_{0} - 1 ) {e}^{- ω {ϕ}_{0} t} ) ,$$

i.e., x evolves from the old steady state x₀ to the new steady state x₀/ϕ₀. Hence, y evolves as

$$y ( t ) = {y}_{0} ( 1 + \frac{{ϕ}_{0} - 1}{1 - \frac{ω {ϕ}_{0}}{κ}} ( {e}^{- ω {ϕ}_{0} t} - {e}^{- κ t} ) ) ,$$

i.e., as two competing exponential decays that produce a transient increase in y, followed by a return to y₀. We use this mechanism as the explanation for the transient effect of Sclerostin antibodies on bone formation, where the role of x is played by the pre-osteoblasts and the role of y is played by the osteoblasts.

1. A method for optimizing a therapy for osteoporosis, comprising:
(a) generating in silico a plurality of virtual patients, wherein each of said virtual patients comprises a mathematical construct for modeling progression of osteoporosis via simulation of bone remodeling, said mathematical construct comprising a plurality of dynamic mathematical relations defining time-dependent evolution of at least one of a cell density variable or concentration variable associated with any of pre-osteoblasts, osteoblasts, preosteoclasts, osteoclasts, osteocytes, a bone resorption signal, sclerostin, estrogen, bone density and bone mineral content and employs a processor to determine time-variation of at least one of bone mineral density and bone mineral fraction based on at least a portion of said time-dependent variables,
(b) applying a simulated therapy to said plurality of virtual patients, and
(c) using said virtual patients to determine an effect of said simulated therapy on progression of osteoporosis.

2. The method of claim 1, further comprising:
(d) adjusting the simulated therapy by adjusting at least one of said parameters of at least one of said dynamic mathematical relations,
(e) applying said adjusted simulated therapy to said virtual patients,
(f) determining an effect of said adjusted simulated therapy on the progression of osteoporosis.

3. The method of claim 2, further comprising repeating steps d)-f) so as to optimize said simulated therapy.

4. The method of claim 3, wherein at least one parameter of at least one of said dynamic mathematical relations is determined based on physiological data collected from a population of actual patients.

5. The method of claim 4, wherein said physiological data comprises any of measured bone mineral density and measured serum concentration of one or more bone turnover markers.

6. The method of claim 5, wherein said one or more bone turnover markers comprise any of N-terminal propeptide of type I procollagen (PINP), bone-specific alkaline phosphatase (BSAP), and C-terminal telopeptide (CTX)

7. The method of claim 2, wherein said simulated therapy comprises virtually administering at least one medication to said virtual patients.

8. The method of claim 7, wherein said at least one medication comprises a combination of two or more medications.

9. The method of claim 6, wherein said step of using said virtual patients to determine the effect of the simulated therapy on the progression of osteoporosis comprises utilizing pharmacokinetic data associated with said at least one medication administered to said virtual patients.

10. The method of claim 7, wherein said step of using virtual patients to determine the effect of the simulated therapy on the progression of osteoporosis comprises selecting a regimen for administration of said at least one medication to said virtual patients.

11. The method of claim 7, wherein said step of adjusting said simulated therapy comprises adjusting a dosage of said at least one medication administered to the virtual patients.

12. The method of claim 7, wherein said step of adjusting said simulated therapy comprises adjusting a dosing regimen of said at least one medication administered to the virtual patients.

13. The method of claim 1, wherein at least one of said virtual patients is configured to simulate an effect of a disease on the progression of the osteoporosis.

14. The method of claim 13, wherein said disease is multiple myeloma.

15. The method of claim 14, wherein said at least one virtual patient simulates the effect of multiple myeloma on the progression of osteoporosis by adjusting at least one of the following rates: a birth rate, a differentiation rate or cell death rate of at least one of pre-osteoblasts, pre-osteoclasts, osteoblasts, osteoclasts, and osteocytes.

16. The method of claim 15, wherein said step of adjusting at least one of said rates comprises defining a mathematical relation indicative of time variation of said rate after onset of said disease.

17. The method of claim 1, wherein at least one of said virtual patients is configured to simulate an effect of glucocorticoid-induced osteoporosis.

18. The method of claim 17, wherein said at least one virtual patient simulates the effect of said disease on the progression of osteoporosis by adjusting at least one of the following rates: a birth rate, a differentiation rate, an amplification factor or cell death rate of at least one of pre-osteoblasts, pre-osteoclasts, osteoblasts, osteoclasts, and osteocytes.

19. The method of claim 1, further comprising adjusting at least one of said variables to simulate an effect of a change in physiological condition on the progression of osteoporosis.

20. The method of claim 19, wherein said physiological condition is onset of menopause.

21. The method of claim 20, wherein said variable comprises estrogen concentration.

22. The method of claim 21, further comprising providing a time-dependent relation defining post-menopausal decline of estrogen as a function of time.

23. The method of claim 19, wherein said physiological condition is caused by aging.

24. The method of claim 23, wherein said variable associated with said age-dependent physiological condition comprises serum concentration of the sclerostin.

25. The method of claim 24, further comprising providing a relation defining time-dependent increase in the serum concentration of the sclerostin after a selected age threshold.

26. The method of claim 1, further comprising adjusting at least one of said dynamic variables to simulate an effect of application of a therapy on progression of osteoporosis.

27. The method of claim 26, wherein said therapy comprises virtual administration of a RANKL inhibitor and said step of adjusting at least one variable comprises defining a mathematical relation indicative of time variation of any of the pre-osteoclast cell density, the osteoclast cell density and the bone mineral content as a function of RANKL inhibitor administration.

28. The method of claim 26, wherein said therapy comprises virtual administration of a sclerostin inhibitor and said step of adjusting at least one variable comprises defining a mathematical relation indicative of time variation of the sclerostin concentration as a function of Sclerostin inhibitor administration.

29. The method of claim 26, wherein said therapy comprises virtual administration of bisphosphonates and said step of adjusting at least one variable comprises defining a mathematical relation indicative of time variation of the osteoclasts cell density as a function of enhanced apoptosis rate due to administration of bisphosphonates.

30. The method of claim 1, further comprising adjusting at least one of said variables to simulate an effect of a physical activity on any of treatment and progression of osteoporosis.

31. The method of claim 30, wherein said at least one variable associated with the physical activity comprises a concentration of the sclerostin.

32.-64. (canceled)
